(19) United States
(12) Patent Application Publication
(10) Pub. No.: US 2005/0038461 A1
(43) Pub. Date: Feb. 17, 2005

(54) Tongue squeegee

(76) Inventor: Kyle Montague Phillips, Bradenton, FL (US)

Correspondence Address: Kyle M. Phillips, 4622 72nd Court east, Bradenton, FL, 34203, US

(21) Appl. No.: 10/419,555

(22) Filed: Aug. 12, 2003

(51) Int. Cl. A61F 9/00

(52) U.S. Cl. 606/161

(57) ABSTRACT

The Tongue Squeegee is a simple modification to a regular tooth brush to aid in the process of tongue cleaning. A thin textured rubber insert or flap is made into or adhered to the underside of a regular toothbrush. The user would then rub the Tongue Squeegee against his tongue whenever he brushed his teeth to aid in removing pathogens and other germs. This would improve oral hygiene on a whole every time we brush or teeth.

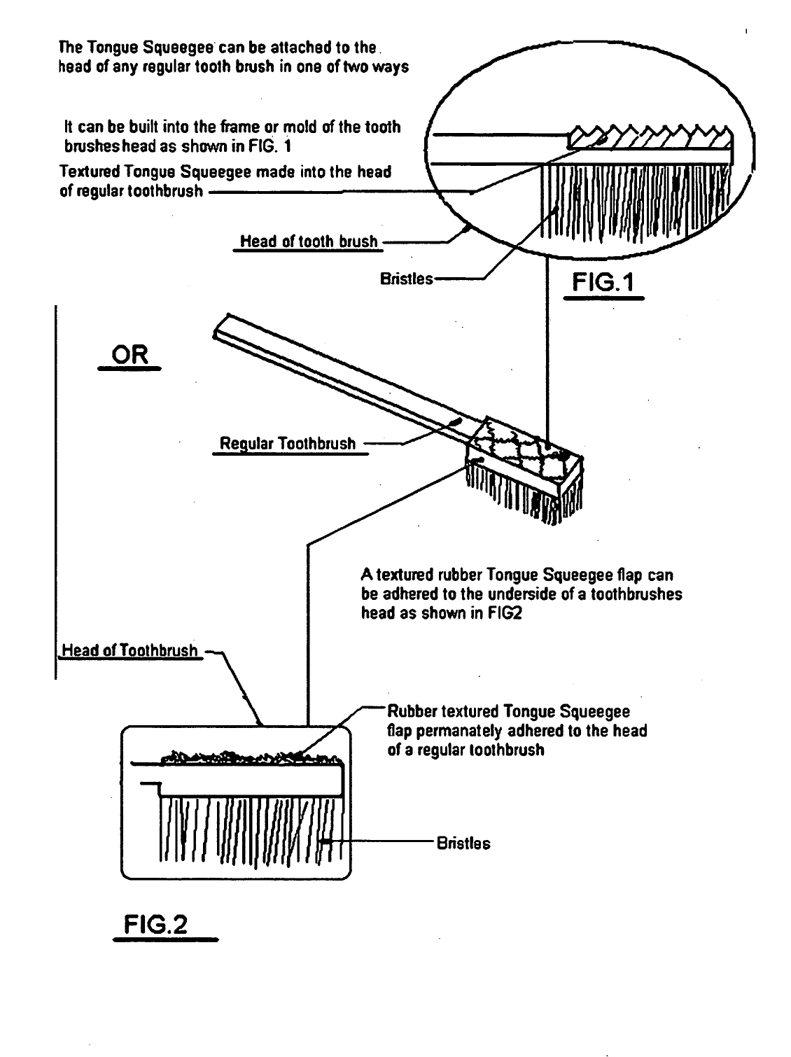

### BACKGROUND OF INVENTION

I had always thought there had to be a way to improve how well a toothbrush functions without resorting to expensive electrical or battery operated products. The task of tongue scrubbing can be made simpler and more hygienic yet still remain inexpensive and uncomplicated by the introduction of a cheap textured rubber flap called the Tongue Squeegee.

### DESCRIPTION OF INVENTION

The Tongue Squeegee would consist of a regular toothbrush which would feature a brushing head upon one end. Resting upon the underside of the brushing head would be a molded section of textured rubber which would be employed to clean the surface of the tongue. This oral care tool would be positioned atop the tongue to remove germs and debris by gently, but firmly, moving it about to scrub the surface of the tongue. This would loosen the pathogens and allow them to be removed by rinsing. It would be possible to produce the Tongue Squeegee in sizes for adults and children.

### PURPOSE OF INVENTION

Oral hygiene.

1. The Tongue Squeegee is a rubber insert or flap witch is either made into or adhered to the underside of the head of any regular toothbrush to aid in the process of oral hygiene i.e. tongue cleaning.
